(19) United States
(12) Patent Application Publication
(10) Pub. No.: US 2008/0306419 A1
(43) Pub. Date: Dec. 11, 2008

(54) Conformable Structures

(76) Inventor: Edward Timothy Bishop, Northamptonshire (GB)

Correspondence Address: AMSTER, ROTHSTEIN & EBENSTEIN LLP, 90 PARK AVENUE, NEW YORK, NY, 10016, US

(21) Appl. No.: 11/659,513

(22) PCT Filed: Aug. 8, 2005
(86) PCT No.: PCT/GB2005/003108

§ 371 Date: Jan. 11, 2008

(30) Foreign Application Priority Data
Aug. 6, 2004 (GB) 0417587.3

(51) Int. Cl. A61F 5/01; A61G 1/04; A47C 7/00

(52) U.S. Cl. 602/6; 5/625; 297/463.2

(57) ABSTRACT

A conformable cushion comprising: a fluid reservoir defined by walls, at least one of which is flexible; electro-rheological fluid located in the reservoir; and a pair of electrodes contacting the electro-rheological fluid whereby an electrical field may be applied across the electro-rheological fluid so as to increase the viscosity thereof.

This invention relates to conformable structures containing electro-rheological materials.

Electro-rheological materials are materials whose rheological properties change when an electric field is applied. Typically the materials behave as fluids in the absence of an electric field. When an electric field is applied the materials' viscosity and shear stress at yield increase.

A number of applications have been proposed for electro-rheological fluids. These include use in clutches, brakes, hydraulic valves and dampers for use in applications such as engine mounts, suspension shock absorbers and seat supports. (See for example U.S. Pat. No. 6,105,420).

In a number of fields situations arise where it is desirable to have an object conform to a desired shape and lock in that configuration.

One example is medical splints. When a patient is injured it is often desirable to fix part of their body in position to prevent further injury and to encourage healing. Inflatable splints are available. They are wrapped around the injured body part and can be inflated to impose an inward force on the body part, holding it in place. However, the force required to keep the body part fixed in place can high, which can cause additional problems such as limiting the blood supply to the body part.

Another example is in seats. People want their seats to conform to their body shape in order to improve comfort. This is normally achieved by making the seat of elastic material such as foam, which compresses to match the contours of the occupant. However, as the occupant moves in the seat the foam expands or compresses to accommodate the motion. As a result, it does little to help hold the occupant in place. Therefore for applications where more support is needed, such as seats for sports cars, the seats are made to a complex pattern including side panels that can restrain the occupant. In order to accommodate occupants of different sizes the resulting seat needs to have a large range of adjustments. This greatly increases the cost of the seat. For critical applications such as racing car seats, foam can be custom-shaped to suit an individual occupant. This is expensive.

Similar issues apply to medical stretchers and beds. The face of a medical stretcher on which a patient is to lie usually has some degree of elasticity which allows it to conform somewhat to the patient's shape. However, due to that elasticity it does not restrain the movement of the patient, which may be desirable in order to prevent further injury. Furthermore, such a stretcher applies pressure unevenly to the patient. Greatest pressure is applied by the stretcher to parts of the patient's body that protrude most. This can cause particular problems for victims of burns.

There is therefore a need for improved conformable structures.

According to the present invention there is provided a conformable cushion and other articles as set out in the accompanying claims.

The present invention will now be described by way of example, with reference to the drawings.

In the drawings:

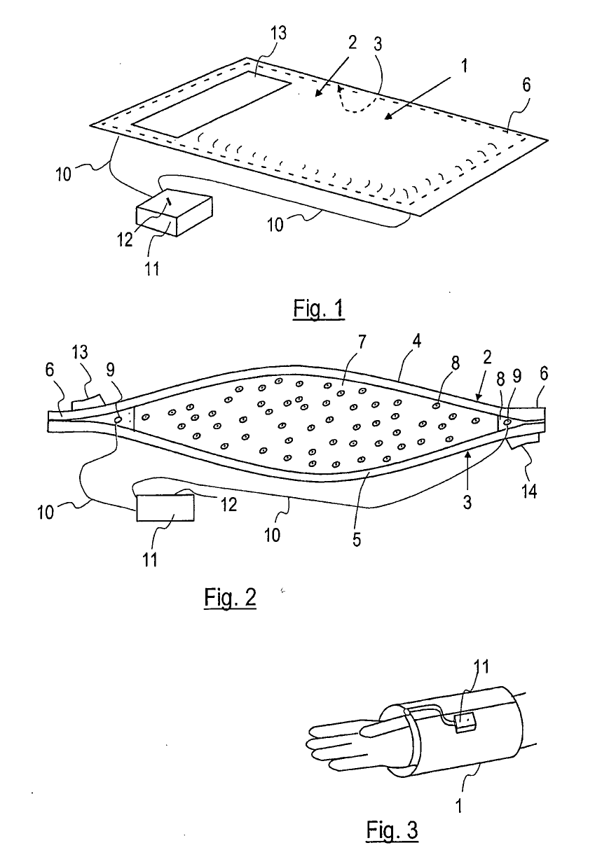

FIG. 1 shows a medical splint in an unrolled configuration;

FIG. 2 shows a cross-section through the splint of FIG. 1;

FIG. 3 shows the splint of FIG. 1 deployed around a limb;

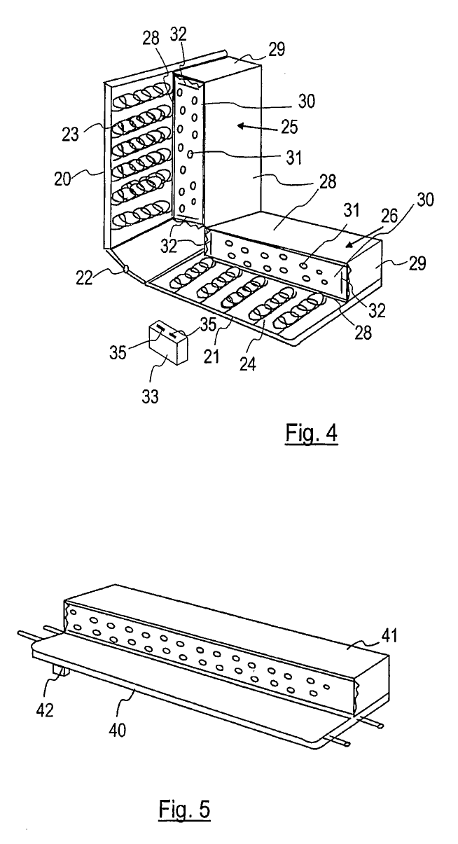

FIG. 4 shows a cut-away view of a vehicle seat; and

FIG. 5 shows a cut-away view of a medical stretcher.

The figures show examples of conformable structures. The structures have cushions comprising flexible-walled reservoirs containing electro-rheological fluid.

A flexible wall of the cushion can be placed against an object so as to conform the surface of the reservoir and the fluid within it to the object. Then an electrical field can be applied to the fluid so as to increase its viscosity and to resist further flow of the fluid. Subsequently the field can be removed, releasing the fluid to adopt another shape.

Electro-rheological fluid generally comprises a carrier liquid in which particles are dispersed. Additives may be included to improve the performance of the liquid. The liquid is a dielectric and could, for example, be an oil. The particles can, for example, be based on silica, zeolites, gum Arabic, formaldehyde polymer, active carbon, poly(acenequinone) radical (PAQR) polymers, polyeurethane polymers or surface treated carbon. When no electrical field is applied the particles are free to move in the fluid and the fluid has a relatively low viscosity. When an electrical field is applied across the fluid the particles take on an ordered structure which resists flow, giving the fluid a relatively high viscosity and a relatively high shear stress at yield. By selection of the materials, the particle loading and any additives, the viscosity of the fluid when no field is applied, and the Theological response of the fluid to the application of a field can be tailored for a desired application.

FIG. 1 shows an example of a medical splint. The splint comprises a cushion 1 defined by a reservoir for electro-rheological fluid. The cushion has a planar shape defined by two major side walls 2, 3 which are joined together at their edges. Each side wall is constituted of a sheet 4, 5 of material that is flexible, impermeable and electrically non-conductive. The sheets are joined together by a weld 6 which runs around the periphery of the reservoir. Inside the reservoir is a sheet 7 of electrically non-conductive, flexible, open cell foam, which is bonded to the walls 2, 3 of the reservoir. The reservoir contains an electro-rheological fluid 8, which is impregnated into the foam 7. At opposite ends of the reservoir are electrodes 9 in contact with the fluid 8. These are connected via cables 10 to a power supply 11, which can apply an electrical field between the electrodes 9 on actuation of switch 12. The power supply could contain a battery as a source of electrical power.

The electro-rheological fluid and the power supply are selected so that the fluid can flow readily when no electrical field is imposed, but so that the fluid becomes substantially rigid when an electrical field is imposed by the power supply.

A fastening arrangement is provided on the surface of the cushion to allow it to be fixed in place on a patient. In the embodiment shown in the figures the fastening arrangement comprises complementary sheets of hook-and-loop fasteners 13, 14 which are attached on opposite sides of the cushion. Those sheets can be locked together when the splint is wrapped around a patient's limb, as shown in FIG. 3. One example of an alternative fastening arrangement is straps that wrap around the splint once it is in place.

In use, the switch 12 is moved to the “off” position to release the electrical field between the electrodes 9, allowing the fluid to flow readily. Then the splint is wrapped around a body part of a patient and fixed in place by engaging one sheet 13 of the hook-and-loop fastener against the other sheet 14. This configuration is illustrated in FIG. 3. The splint is wrapped around the body part sufficiently tightly that the fluid in the reservoir conforms to the shape of the body part. Then the switch 12 is moved to the “on” position to apply an electrical field between the electrodes 9. This causes the fluid to become rigid, locking the body part in place. When the splint is to be released, the switch is returned to the “off” position and the splint is removed. The splint can be re-used, and can then adopt the shape of another body part.

The cushion 1 could be shaped to provide better performance in certain situations. For example, if the splint were to be used as a neck brace then it could have anatomically shaped shoulder, neck and head regions, which might wrap around the patient separately. The shaping of the cushion could be done by suitable design of the shapes of the sheets 4, 5 that define the major walls of the reservoir, by the presence of side walls running between the sheets 4, 5, and by the shaping of the foam 7 between the sheets.

The power supply 11 and/or the cables 10 could attach to the reservoir (as shown in FIG. 3) to allow the reservoir to be moved more easily.

The foam 7 is advantageous in that it resists compression of the cushion, inhibiting the complete displacement of the fluid from regions under the greatest compression. Subsequent expansion of the foam is resisted when the fluid has been locked by the application of an electrical field. Other materials that are elastically resistant to compression but allow electrical continuity of the fluid between the electrodes could be used. One example is pillars of impermeable elastic material such as rubber, located between the sheets. Such pillars could be bonded to one or both sheets to keep them in place.

The foam 7 could be bonded to both, either or none of the sheets. However, when the foam is bonded to both sheets it also has the advantage that it keeps the sheets 4, 5 from moving outward when the fluid has been locked. Other constructions could be used to achieve a similar result. For example, discrete strips of material could extend across the reservoir in various places and be bonded to the sheets 4, 5.

Instead of the electrodes being at opposite ends of the reservoir, as illustrated in FIG. 1, they could be provided on the major faces of the reservoir. Sheets of electrically conductive material could be bonded to the interior walls of the sheets 4, 5 and connected to the cables 10 to act as the electrodes. The sheets of conductive material could be laminated on to the whole surface of the sheets 4, 5 and kept apart by a spacer at the weld. Alternatively, the sheets of conductive material could be inset from the edges of the sheets 4, 5 so that they do not meet at the weld.

The splint of FIGS. 1 to 3 can also provide heat for accelerating the recovery of the patient. Heating coils 15 are set into the foam 7 and can be supplied with power from the power supply 11. The heating coils could alternatively be bonded to one or both of the sheets 4, 5. The heating coils contain an inner core of resistive wire surrounded by an insulating layer to prevent shorting with the fluid. A range of electro-rheological fluids generate heat on application of an electrical field through the fluid—particularly when the fluid has reached maximum viscosity. Thus the fluid 8 could be capable of generating heat on application of an electrical field through the fluid itself.

The cushion could be divided into a number of independent zones by means of electrically insulating baffles. Each zone would have its own pair of electrodes and could then be independently controlled. Separate coils 15 could be provided in each region.

The principles of construction described above for the cushion of the splint shown in FIGS. 1 to 3 can be applied to other applications. Some examples will be described below. In other applications one or more walls of the cushion could be solid, provided the cushion has at least one flexible wall

FIG. 4 illustrates an example of a vehicle seat. The seat comprises a frame having a back section 20 and a base section 21. The back section and the base section are joined by a hinge arrangement 22 in the normal way, so that the back can be hinged relative to the base. Sprung frames 23, 24 are attached to the back and base sections respectively. On each of the sprung frames is a cushion 25, 26. The cushions are covered with a seating fabric (not shown), which could optionally be backed with a thin foam layer to smooth out its shape.

The cushions 25, 26 are similar to the reservoir 1 of FIGS. 1 to 3. Each cushion is defined by sheets of material that is flexible, impermeable, and electrically non-conductive. The sheets are welded together. In this design of cushion there are sheets 28 defining major faces of the cushion, and sheets 29 defining edge faces.

Inside each cushion is a sheet 30 of electrically non-conductive, flexible, open cell foam, which is bonded to the sheets 29. Each cushion contains an electro-rheological fluid 31, which is impregnated into the foam 29. At opposite ends of each cushion are electrodes 32 in contact with the fluid 31. These are connected via cables (not shown) to control unit 33, which can apply an electrical field between the electrodes 32 on actuation of a respective switch 35. The power supply could be got from the vehicle's electrical circuits.

The electro-rheological fluid and the power supply are selected so that the fluid can flow when no electrical field is imposed, but so that the fluid becomes substantially rigid when an electrical field is imposed by the power supply. The viscosity of the fluid when no field is imposed can be selected based on the application.

In use, an occupant sits in the seat with the power to the cushions switched off by means of switches 35 for sufficient time to allow the fluid 31 to conform to his shape. Then the switches are actuated so as to apply an electrical field across the fluid, locking the fluid in place. The seat is thus moulded to the shape of the occupant. When another person wishes to use the seat, he can repeat the process to have the seat mould to his body.

The switches 35 could be push-button switches and the control unit 33 could be arranged to automatically release the electrical field for a pre-determined time on actuation of the switches, and then to reapply the field. The predetermined time could be set based on the viscosity of the fluid when no field is applied and the elastic modulus of the foam 30, so as to automatically allow sufficient time for the fluid to conform to an occupant but not so much time that the occupant will have displaced the fluid excessively.

The seat could have multiple such cushions on the base or back. The seat could have cushions on side walls of the base and back and in the headrest.

FIG. 5 illustrates an example of a medical stretcher. The stretcher has a frame 40 on which is a cushion 41 analogous to those of FIGS. 1 to 4. The cushion can be made to conform to the body shape of a person lying on the cushion by suitable operation of the power supply 42. Such a stretcher has the advantage that it can help to resist movement of the patient as the stretcher is moved.

Similar principles could be applied to other products such as beds, pillows, independent cushions, saddles, shoe footbeds and packaging.

The applicant hereby discloses in isolation each individual feature described herein and any combination of two or more such features, to the extent that such features or combinations are capable of being carried out based on the present specification as a whole in light of the common general knowledge of a person skilled in the art, irrespective of whether such features or combinations of features solve any problems disclosed herein, and without limitation to the scope of the claims. The applicant indicates that aspects of the present invention may consist of any such feature or combination of features. In view of the foregoing description it will be evident to a person skilled in the art that various modifications may be made within the scope of the invention.

1. A conformable cushion comprising:
a fluid reservoir defined by walls, at least one of which is flexible;
electro-rheological fluid located in the reservoir; and
a pair of electrodes contacting the electro-rheological fluid whereby an electrical field may be applied across the electro-rheological fluid so as to increase the viscosity thereof.

2. A conformable cushion as claimed in claim 1, comprising connecting means extending through the reservoir from the flexible wall to an opposing wall of the reservoir for restraining movement of the flexible wall away from the opposing wall.

3. A cushion as claimed in claim 2, wherein the walls of the fluid reservoir include two major walls, one of the major walls being the said flexible wall and the other of the major walls being the opposing wall.

4. A cushion as claimed in claim 3, wherein the said other of the major walls is flexible.

5. A cushion as claimed in claim 4, comprising fixing means for fixing the cushion around a body part.

6. A cushion as claimed in claim 5, wherein the fixing means is a hook-and-loop fastening arrangement.

7. A cushion as claimed in claim 6, wherein one part of a hook-and-loop fastening arrangement is located on the exterior of one of the major walls, and the counterpart of the hook-and-loop fastening arrangement is located on the exterior of the other of the major walls.

8. A cushion as claimed in claim 2, comprising a resilient means for resisting compression of the cushion.

9. A cushion as claimed in claim 8, wherein the spacing means and the connecting means are provided by the same component(s).

10. A cushion as claimed in claim 8, wherein the resilient means is a foam.

11. A cushion as claimed in claim 10, wherein the resilient means is an open cell foam.

12. A cushion as claimed in claim 10, wherein the foam occupies substantially the whole of the reservoir,

13. A cushion as claimed in claim 1, comprising a heater for generating heat on the application of an electric current thereto.

14. A cushion as claimed in claim 1, further comprising a power supply arranged for applying an electric field between the electrodes.

15. (canceled)

16. A splint comprising a cushion as claimed in claim 1.

17. A seat comprising a cushion as claimed in claim 1.

18. A vehicle comprising a seat as claimed in claim 1.

19. A stretcher comprising a cushion as claimed in claim 1.
